(19) United States
(12) Patent Application Publication
(10) Pub. No.: US 2023/0298165 A1
(43) Pub. Date: Sep. 21, 2023

(54) NEUROIMAGING METHODS AND SYSTEMS

(71) Applicants: INSTIT NATIONAL DE LA SANTE ET DE LA RECHERCHE MEDICALE (INSERM), Paris (FR); UNIVERSITE DE CAEN NORMANDIE, CAEN (FR)

(72) Inventors: Robin DE FLORES, CAEN (FR); Gaëlle CHETELAT, CAEN (FR); Brigitte LANDEAU, CAEN (FR); Sophie DAUTRICOURT, CAEN (FR)

(21) Appl. No.: 18/006,490

(22) PCT Filed: Jul. 23, 2021
(86) PCT No.: PCT/EP2021/070727

§ 371 Date: Jan. 23, 2023

(30) Foreign Application Priority Data
Jul. 24, 2020 (EP) 20305853.2

(51) Int. Cl. G06T 7/00; A61B 5/055

(52) U.S. Cl. CPC G06T 7/0012; A61B 5/055; G06T 2207/30016; G06T 2207/10088; G06T 2207/20084

(57) ABSTRACT

The invention relates to neuroimaging techniques and more specifically to methods and systems for identifying and/or predicting the occurrence of neurodegenerative diseases (such as Alzheimer's disease) or cognitive impairment in a subject, based on magnetic resonance images (MRI). Anatomical and functional MRI brain images are combined to build functional connectivity maps of one or more medial temporal lobe (MTL) subregions of the brain of a subject. Functional connectivity networks associated to the anterior temporal (AT) and posterior medial (PM) hippocampal network are then identified. A metric based on the degree of functional connectivity in both networks can be used as a tool to detect functional changes associated to cognitive decline caused by neurodegenerative diseases.

### TECHNICAL FIELD

The present invention relates to neuroimaging methods and systems.

The present invention also relates to methods and systems for the prognostic assessment of cognitive decline due to neurodegenerative diseases, such as Alzheimer's disease.

### BACKGROUND

Neurodegenerative diseases affecting the human brain, such as semantic dementia or Alzheimer's disease, can result in cognitive decline and alterations of brain structure and function.

Conventional neuroimaging methods are a useful tool to observe and evidence lesions, loss of function and other symptoms once the neurodegenerative disease has progressed beyond a certain stage of development. However, at this stage, it is generally too late to treat the disease, since the lesions and loss of function are usually irreversible and permanent.

There is thus a need for neuroimaging methods and systems, especially for assessing cognitive decline induced by neurodegenerative diseases at an early stage of development.

### SUMMARY

An object of the present invention is therefore to provide a computer implemented-method comprising:
acquiring a first set of at least one anatomical magnetic resonance image (MRI) of a brain region of a subject and a second set of functional magnetic resonance (fMRI) images of the same brain region of the same subject;
segmenting the at least one image of the first set to highlight specific brain sub-regions of interest;
combining the at least one segmented image of the first set with the corresponding images of the second set to highlight the sub-regions of interest on the second images;
computing, from the functional data of the second set of images, a functional connectivity map for each of the sub-regions of interest;
identifying a neural connectivity network for each of the sub-regions of interest,
calculating a score representative of the functional connectivity of the neural connectivity networks of the sub-regions of interest.

According to other advantageous aspects, the invention may comprise one or more of the following technical features, considered alone or according to all possible combinations:
A connectivity strength value is calculated for each of at least two identified neural connectivity networks, and wherein the calculated score is a ratio between the connectivity strength values of said identified neural connectivity networks.
The identified neural connectivity networks comprise at least the anterior temporal (AT) cortical network of the brain and the posterior medial (PM) cortical network of the brain.
The calculated score is a ratio between the connectivity strength value of the anterior temporal neural connectivity network and the connectivity strength value of the posterior medial neural connectivity network.
The anatomical magnetic resonance images are -weighted magnetic resonance images.
The functional magnetic resonance images are resting-state functional magnetic resonance images.
The images of the first set are segmented using a multi-atlas segmentation algorithm.

According to another aspect, a method of determining the prognosis of a subject suffering from cognitive impairment, comprises
calculating a first score representative of the functional connectivity of neural connectivity networks identified from magnetic resonance images of a brain region of the subject, using a method according to embodiments as previously described;
calculating a second score representative of the functional connectivity of neural connectivity networks identified from additional magnetic resonance images of the brain region of the same subject, using a method according to embodiments as previously described, said additional having been acquired at a later date than the original magnetic resonance images;
comparing the first score with the second score and providing a positive prognosis or a negative prognosis depending on the result of the comparison.

According to another aspect, each score is a ratio of the connectivity strength value of the anterior temporal neural connectivity network over the connectivity strength value of the posterior medial neural connectivity network of the imaged brain, and wherein a negative prognosis is provided if the second score is higher than the first score.

According to another aspect, a computer system is configured to:
acquire a first set of at least one anatomical magnetic resonance images (MRI) of a brain region of a subject and a second set of resting-state functional magnetic resonance (fMRI) images of the same brain region of the same subject;
   segment the at least one image of the first set to highlight specific brain sub-regions of interest;
   combine the at least one segmented image of the first set with the corresponding images of the second set to highlight the sub-regions of interest on the second images;
   combine, from the functional data of the second set of images, a functional connectivity map for each of the sub-regions of interest;
   identify a neural connectivity network for each of the sub-regions of interest,
   calculate a score representative of the functional connectivity of the neural connectivity networks of the sub-regions of interest.

According to another aspect, the computer system is further programmed to implement a method for determining the prognosis of a subject suffering from cognitive impairment, wherein the computer system is configured to compare the calculated score with a predefined threshold, and providing a positive prognosis or a negative prognosis depending on the result of the comparison.

According to another aspect, the computer system is further programmed to implement a method for determining the prognosis of a subject suffering from cognitive impairment, wherein the computer system is configured to:
calculate a second score representative of the functional connectivity of neural connectivity networks identified from additional magnetic resonance images of the brain region of the same subject, using a method according to the invention, said additional magnetic resonance images having been acquired at a later date than the original magnetic resonance images;
compare the score with the second score and providing a positive prognosis or a negative prognosis depending on the result of the comparison.

According to another aspect, the invention relates to a method for establishing a clinical diagnosis based on the determined prognosis.

### BRIEF DESCRIPTION OF THE DRAWINGS

The invention will be better understood upon reading the following description, provided solely as an example, and made in reference to the appended drawings, in which:

FIG. 1 is a simplified representation of an exemplary system for acquiring and processing magnetic resonance images of a brain tissue;

FIG. 2 is a flow chart depicting a neuroimaging method according to embodiments of the invention;

FIG. 3 is an exemplary composite image showing functional magnetic resonance imaging data of a brain in several regions of interest viewed along three cross-sectional geometrical planes, wherein the regions of interest include the perirhinal cortex (PRC, inset A) and the parahippocampal cortex (PHC, inset B);

FIG. 4 is a flow chart depicting an embodiment of a method for the prognostic assessment of cognitive decline using the neuroimaging method of FIG. 2;

FIG. 5 is a flow chart depicting another embodiment of a method for the prognostic assessment of cognitive decline using the neuroimaging method of FIG. 2.

### DETAILED DESCRIPTION OF SOME EMBODIMENTS

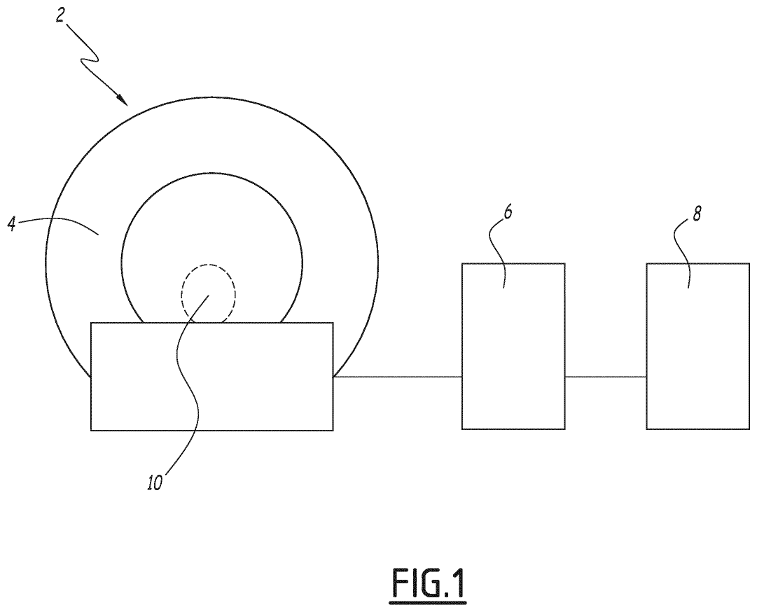

On FIG. 1 there is illustrated an exemplary neuroimaging system 2 comprising a magnetic resonance imaging (MRI) device 4, a first computer system 6 and a second computer system 8.

The MRI device 4 is configured to acquire one or more MRI images of a subject 10, such as a human subject 10, for example to acquire one or more MRI images of brain tissue from the subject 10, such as images of a brain region of the subject 10.

The subject 10 is not part of the MRI device 4 or of the imaging system 2.

According to some embodiments, the first computer system 4 is operatively coupled to the MRI device 4 (e.g., connected through a wired communication link or a wireless communication link) and is configured to operate the MRI device 4.

For example, the first computer system 6 comprises control circuitry configured to acquire anatomical MRI images of a brain region of the patient 10 as well as functional MRI images (fMRI) if the same brain region of the patient 10.

For example, the acquired anatomical magnetic resonance images are T1-weighted magnetic resonance images.

The functional magnetic resonance images may be resting-state functional magnetic resonance images, i.e. MRI images acquired while the subject 10 is resting and is not performing any specific task (such as a movement or a cognitive task).

The acquired MRI images and/or acquired MRI data may be stored in a memory storage device of the first computer system 6 and may be processed and/or downloaded by another computer system, such as the second computer system 8.

The second computer system 8 is operatively coupled to the MRI device 4 (e.g., connected through a wired communication link or a wireless communication link) to the first computer system 6 and is configured to process one or more of the MRI images acquired by the MRI device 4 (and the first computer system 6).

The computer system 8 may comprise control circuitry, such as a processor and a memory storage device, wherein executable instructions and/or control algorithms are stored in order to operate one or more of the methods described thereafter.

The memory device may include, for example, a random access memory (RAM), and/or other forms of memory, such as flash memory, programmable read only memory (PROM), and electronically erasable programmable read only memory (EEPROM), a magnetic storage drive, an optical storage drive, or any appropriate computer-readable data storage device.

The computer system 8 may also comprise a data input interface, including, but not limited to, one or more of the following human machine interface elements: a display screen, a touch-sensitive screen, a keyboard, a pointer, a mouse, a trackpad, a microphone, or the like. The data input interface may also include a wired connector and/or a wireless connection interface for connecting a personal computing device such as a mobile phone or a portable tablet configured to implement a graphical user interface.

This example is shown for illustrative purposes only. In alternative embodiments, the first computer system 6 and the second computer system 8 could be merged into a single computer system. The second computer system 8 could also be used independently from the MRI device 4 and the first computer system 6, e.g. it could be used offsite, in a different location.

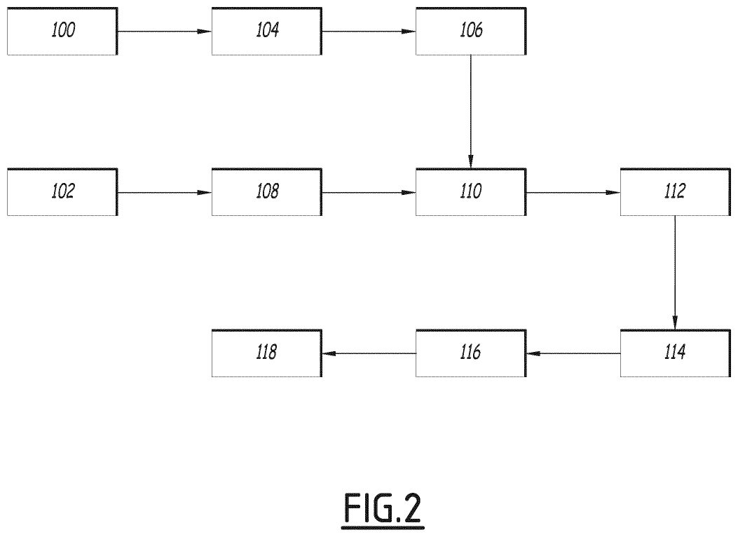

An exemplary neuroimaging method is now described in reference to FIG. 2.

Preferably, this neuroimaging method is implemented by the computer system 8, using the neuroimaging system 2.

At block 100, one or more anatomical magnetic resonance images (first set of images) of a brain region of the subject 10 are acquired, for example using the MRI device 4.

In practice, with modern MRI imaging systems, it is possible to acquire a single image covering the entire brain.

At block 102, a set of functional magnetic resonance images (second set of images) of the same brain region of the same subject 10 is acquired.

For example, the second set of MRI images, or even each set of MRI images, may comprise a plurality of MRI images acquired successively according to a specific time sequence.

Preferably, both first and second sets of images may be acquired by the same MRI device 4.

At block 104, the images of the first set are automatically segmented to highlight specific brain sub-regions of interest.

The sub regions of interest, also known as “seeds”, may correspond to medial temporal lobe (MTL) sub regions of the brain.

In what follows, the sub-regions of interest may be referred to as “regions of interest” when the acquired images encompass the whole brain.

As will be made apparent in what follows, in preferred embodiments, the sub regions of interest include at least the following brain areas of the medial temporal lobe: the perirhinal cortex, and the parahippocampal cortex.

In other embodiments, other brain sub-regions of interest could be selected.

Preferably, during step 104, images are segmented using a multi-atlas segmentation algorithm.

According to a preferred example, the multi-atlas segmentation algorithm can be the Automatic Segmentation of Hippocampal Subfields (ASHS) algorithm described in the following citation:

Xie L, Wisse L E M, Pluta J, et al. Automated segmentation of medial temporal lobe subregions on in vivo T1-weighted MRI in early stages of Alzheimer's disease. Hum Brain Mapp. 2019;40(12):3431-3451. doi:10.1002/hbm.24607.

This algorithm is particularly effective for precisely identifying the regions of interest.

More specifically, this method is more effective at distinguishing brain tissue from the surrounding tissues such as dura mater, and accounts for anatomical variabilities often found in the brain regions of interest.

Other embodiments and implementations are nonetheless possible.

In practice, at the end of the segmentation step (shown separately as block 106), the image(s) from the first set comprise only MRI data for the sub regions of interest. For example, MRI data (such as voxel values) located outside the sub regions of interest has been removed.

Optionally, at block 108, the fMRI images from the second set can be inspected for quality control and can be pre-processed following one or more pre-processing steps, including for example: slice timing correction, distortion correction, spatial normalization, spatial smoothing and temporal filtering.

Overall, these pre-processing steps allow to clean and prepare the acquired data for analyses with the goal of reducing unwanted imaging artefacts.

At block 110, the segmented anatomical image(s) (the first set of images) is (are) combined with the corresponding images of the second set in order to highlight the regions of interest on the functional MRI images.

As a result, the functional data (fMRI data) located inside the regions of interests can be easily accessed, for example during further processing steps. However, in most embodiments, the functional data (fMRI data) located outside the subregions of interest is still present in the underlying functional images.

For example, during step 110, time-dependent data may be selected from the highlighted regions of interest.

In some embodiments, if multiple anatomical images have been acquired during step 100, then each image from the second set of images can be paired with a corresponding image from the first set of images (e.g., an image depicting the same area of the brain).

At block 112, a functional connectivity map is computed from the functional data of the second set of images, for each of the sub-regions of interest.

In the meaning of the present disclosure, “connectivity” refers to a time-dependent correlation between spatially separated regions or sub regions of the brain.

For example, during step 112, the time-dependent data selected from the regions of interest is correlated with the time-dependent data associated to voxels belonging to the other imaged areas of the brain, i.e. regions other than the sub-regions of interest.

At block 114, a neural connectivity network (or cortical network) is automatically identified for each of the sub-regions of interest, using the computed functional connectivity maps.

In the meaning of the present disclosure, a “neural connectivity network” refers to a group of brain regions or brain sub regions that display a synchronized functional connectivity when the subject 10 is in a specific cognitive state, such as a resting state.

For example, only the brain regions for which the functional time-dependent correlation with the selected region of interest is higher than a predefined correlation threshold are deemed to display a synchronized functional connectivity.

In some preferred embodiments, the identified connectivity networks of the medial temporal lobe of the brain comprise at least the anterior temporal (AT) cortical network and the posterior medial (PM) cortical network.

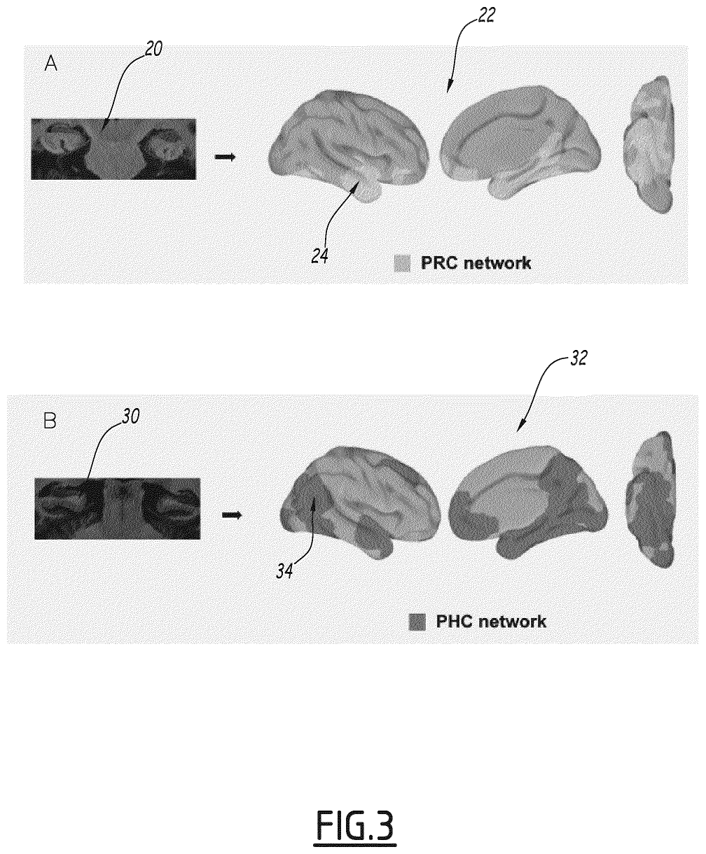

FIG. 3 is an exemplary composite image showing functional magnetic resonance imaging data of a brain in several regions of interest viewed along three cross-sectional geometrical planes.

On insert A), there is illustrated a segmented anatomical MRI image 20, for example generated using the method implemented during step 104, for a first sub region: the perirhinal cortex (PRC).

As explained above, the segmented image 20 may be combined with one or more corresponding functional images from the second set of images in order to ultimately highlight the neural connectivity network in the functional images.

On the resulting reconstructed brain images 22, the identified cortical network (in this case, the AT network) associated to the first subregion of interest is highlighted and designated by the reference sign 24.

Insert B) similarly illustrates a segmented anatomical MRI image 30 for a second sub region: the parahippocampal cortex (PHC). On the resulting reconstructed brain images 32, the identified cortical network (in this case, the PM network) associated to the second subregion of interest is highlighted and designated by the reference sign 34.

Referring back to FIG. 2, in some embodiments, at block 116, a connectivity strength value may be calculated for each of at least two identified neural connectivity networks, or more generally for each of the identified connectivity networks.

At block 118, a score representative of the functional connectivity of the neural connectivity networks of the sub-regions of interest is automatically computed.

In some embodiments, the calculated score is a ratio between the connectivity strength values of said identified neural connectivity networks calculated during step 116.

In that case, the calculated score may be defined as the ratio between the connectivity strength value of the anterior temporal neural connectivity network and the connectivity strength value of the posterior medial neural connectivity network.

More precisely, the calculated score may be defined as the ratio of the connectivity strength value of the anterior temporal neural connectivity network over the connectivity strength value of the posterior medial neural connectivity network.

For example, this ratio can be defined by the following formula:

S=AT_s/PM_s

where “S” is the calculated score, “AT_s” is the connectivity strength value of the anterior temporal neural connectivity network, and “PM_s” is the connectivity strength value of the posterior medial neural connectivity network.

Other embodiments and implementations are nonetheless possible.

In many alternative embodiments, the method steps described above could be executed in a different order. One or more method steps could be omitted or replaced by equivalent steps. One or more method steps could be combined or dissociated into different method steps. The disclosed embodiment is not intended to be limiting and does not prevent other methods steps to be executed without departing from the scope of the claimed subject matter.

The inventors have determined that the degree of functional connectivity in different connectivity networks is used to define a metric (the connectivity score) that can be advantageously used as a tool to detect functional changes associated to cognitive decline caused by neurodegenerative diseases, preferably while the disease is still at an early stage of development.

More specifically, an increase of connectivity strength in the anterior temporal neural connectivity network, and a decrease of connectivity strength in the posterior medial neural connectivity network, can both be used as evidence of cognitive and physiological changes correlating with the occurrence of neurodegenerative diseases, such as Alzheimer's disease.

For example, an increase of connectivity in the anterior temporal neural connectivity network may be associated with an increase of tau protein accumulation, while a decrease of connectivity in the posterior medial neural connectivity network may be associated to increased amyloid pathology.

In that case, an increase of the proposed connectivity score (either an increase over time for successive measurements in a same subject 10, or an increase over a predefined threshold, e.g. computed for a specific population group statistically representative of the subject 10) can be used to evidence the progression of a neurodegenerative disease.

Therefore, the connectivity score can be used as part of an early diagnosis method to detect the occurrence of neurodegenerative diseases or cognitive impairment, and/or to measure or quantify the advancement of said disease.

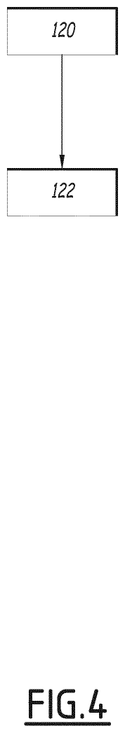

On FIG. 4 is illustrated an exemplary method for determining the prognosis of a subject suffering from cognitive impairment.

Preferably, embodiments of this method use the connectivity score calculated by embodiments of the neuroimaging method disclosed above.

This method may be implemented by the computer system 8.

At block 120, a score representative of the functional connectivity of neural connectivity networks identified from magnetic resonance images of a brain region of the subject is calculated, for example using steps 100 through 118.

At block 122, the calculated score is compared with a predefined threshold. Then, a positive prognosis or a negative prognosis is automatically provided, depending on the result of the comparison.

The predefined threshold may be computed in advance for a specific population group statistically representative of the subject 10.

For example, if the calculated score exceeds a predefined threshold, then a negative prognosis is provided. Conversely, if the calculated score remains below the predefined threshold, then a positive prognosis is provided.

This is especially applicable if the connectivity score is defined as the ratio of the connectivity strength value of the anterior temporal neural connectivity network over the connectivity strength value of the posterior medial neural connectivity network, as envisioned in some embodiments described above.

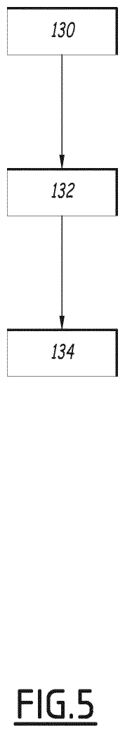

On FIG. 5 there is illustrated another exemplary method for determining the prognosis of a subject suffering from cognitive impairment, using the connectivity score calculated by embodiments of the neuroimaging method disclosed above.

Preferably, embodiments of this method use the connectivity score calculated by embodiments of the neuroimaging method disclosed above.

This method may be implemented by the computer system 8.

At block 130, a first score representative of the functional connectivity of neural connectivity networks identified from magnetic resonance images of a brain region of the subject is computed.

For example, the first and second sets of MRI images (named “original images” in what follows) used to compute the first score may be acquired at a first date.

At block 132, a second score representative of the functional connectivity of neural connectivity networks identified from additional magnetic resonance images of the brain region of the same subject, using once again the same method as described above.

For example, the first and second sets of MRI images used to compute the second score have been acquired at a later date than the first date at which the original magnetic resonance images have been acquired.

At block 134, the first score is compared to the second score. Then, a positive prognosis or a negative prognosis is provided, depending on the result of the comparison.

For example, if each connectivity score is defined as the of the connectivity strength value of the anterior temporal neural connectivity network over the connectivity strength value of the posterior medial neural connectivity network of the imaged brain, then a negative prognosis is provided 134 if the second score is higher than the first score.

It is thus possible to detect changes in cognitive performance in a subject over time.

Other embodiments and implementations are nonetheless possible.

The embodiments and alternatives described above may be combined with each other in order to create new embodiments of the invention.

1. A computer-implemented method, comprising:
acquiring a first set of at least one anatomical magnetic resonance image of a brain region of a subject and a second set of functional magnetic resonance images of the same brain region of the same subject;
segmenting the at least one image of the first set to highlight specific brain sub-regions of interest;
combining the at least one segmented image of the first set with the corresponding images of the second set to highlight the sub-regions of interest on the second images;
computing, from the functional data of the second set of images, a functional connectivity map for each of the sub-regions of interest;
identifying a neural connectivity network for each of the sub-regions of interest, and
calculating a score representative of the functional connectivity of the neural connectivity networks of the sub-regions of interest.

2. The method of claim 1, wherein a connectivity strength value is calculated for each of at least two identified neural connectivity networks,
and wherein the calculated score is a ratio between the connectivity strength values of said identified neural connectivity networks.

3. The method of claim 1, wherein the identified neural connectivity networks comprise at least the anterior temporal cortical network and the posterior medial cortical network.

4. The method of claim 2, wherein the calculated score is a ratio between the connectivity strength value of the anterior temporal neural connectivity network and the connectivity strength value of the posterior medial neural connectivity network.

5. The method according to claim 1, wherein the anatomical magnetic resonance images are T1-weighted magnetic resonance images.

6. The method according to claim 1, wherein the functional magnetic resonance images are resting-state functional magnetic resonance images.

7. The method according to claim 1, wherein the images of the first set are segmented using a multi-atlas segmentation algorithm.

8. A method for determining the prognosis of a subject suffering from cognitive impairment, comprising:
calculating a score representative of the functional connectivity of neural connectivity networks identified from magnetic resonance images of a brain region of the subject, using a the computer-implemented method according to claim 1;
comparing the calculated score with a predefined threshold, and
providing a positive prognosis or a negative prognosis depending on the result of the comparison.

9. A method of determining the prognosis of a subject suffering from cognitive impairment, comprising
calculating a first score representative of the functional connectivity of neural connectivity networks identified from magnetic resonance images of a brain region of the subject, using a the computer-implemented method according to claim 1;
calculating a second score representative of the functional connectivity of neural connectivity networks identified from additional magnetic resonance images of the brain region of the same subject, using the computer-implemented method according to claim 1, said additional magnetic resonance images having been acquired at a later date than the original magnetic resonance images;
comparing the first score with the second score and providing a positive prognosis or a negative prognosis depending on the result of the comparison.

10. The method of claim 9, wherein each score is a ratio of the connectivity strength value of the anterior temporal neural connectivity network over the connectivity strength value of the posterior medial neural connectivity network of the imaged brain,
and wherein a negative prognosis is provided if the second score is higher than the first score.

11. A method for establishing a clinical diagnosis based on the prognosis determined with the method of claim 9.

12. A computer system, configured to:
acquire a first set of at least one anatomical magnetic resonance images of a brain region of a subject and a second set of resting-state functional magnetic resonance images of the same brain region of the same subject;
segment the at least one image of the first set to highlight specific brain sub-regions of interest;
combine the at least one segmented image of the first set with the corresponding images of the second set to highlight the sub-regions of interest on the second images;
combine, from the functional data of the second set of images, a functional connectivity map for each of the sub-regions of interest;
identify a neural connectivity network for each of the sub-regions of interest,
calculate a score representative of the functional connectivity of the neural connectivity networks of the sub-regions of interest.

13. The computer system of claim 12, wherein the computer system is further programmed to implement a method for determining the prognosis of a subject suffering from cognitive impairment,
wherein the computer system is configured to compare the calculated score with a predefined threshold, and to provide a positive prognosis or a negative prognosis depending on the result of the comparison.

14. The computer system of claim 12, wherein the computer system is further programmed to implement a method for determining the prognosis of a subject suffering from cognitive impairment, wherein the computer system is configured to:
calculate a second score representative of the functional connectivity of neural connectivity networks identified from additional magnetic resonance images of the brain region of the same subject, said additional magnetic resonance images having been acquired at a later date than the original magnetic resonance images; and
compare the score with the second score and provide a positive prognosis or a negative prognosis depending on the result of the comparison.

15. A method for establishing a clinical diagnosis based on the prognosis determined with the method of claim 10.
